(19) United States
(12) Patent Application Publication
(10) Pub. No.: US 2006/0122532 A1
(43) Pub. Date: Jun. 8, 2006

(54) Refrigerator with body fat meter

(75) Inventors: Chao-Fa Lee, Taipei City (TW); Shih-Jung Chang, Taipei City (TW); Cheng-Hsing Kuo, Taipei City (TW); Shu-Ting Liao, Taipei City (TW)

Correspondence Address: BACON & THOMAS, PLLC, 625 SLATERS LANE, FOURTH FLOOR, ALEXANDRIA, VA, 22314, US

(73) Assignee: Tatung Co., Ltd., Taipei City (TW)

(21) Appl. No.: 11/212,568

(22) Filed: Aug. 29, 2005

(30) Foreign Application Priority Data
Dec. 8, 2004 (TW) 093219775

(51) Int. Cl. A61B 5/05

(52) U.S. Cl. 600/547

(57) ABSTRACT

A refrigerator is disclosed. The refrigerator with a body fat meter calculates a body fat rate according to an impedance of a human body when the user touches both of the body fat detecting buttons, displays the body fat rate on a screen, and keeps a historical record of the detected body fat rate. According to the historical record of the detected body fat rate, the user may be aware of the increase of the body fat rate.

### BACKGROUND OF THE INVENTION

1. Field of the Invention

The present invention generally relates to a refrigerator and, more particularly, to a refrigerator with a body fat meter.

2. Description of the Related Art

Body fat rate is a new index to evaluate a health condition of human being. To prevent suffering from diseases, a body fat meter is becoming an indispensable medical appliance in every family. Most domestic body fat meters, however, stand alone and display a detected body fat rate without transmitting the detected body fat rate to other electric appliances, and thus it is inconvenient for use not to integrate the body fat meter and other electric appliances.

A technology of a conventional refrigerator is focused on refrigeration and energy-saving without other additional functions. Because the conventional refrigerator is not capable of detecting the body fat rate, users cannot be aware of that some food in the conventional refrigerator may be harmful to their health. As a result, the conventional refrigerator without body fat detecting function is out of time. Thus, there is a general need for a refrigerator with a body fat meter.

### BRIEF SUMMARY OF THE INVENTION

The present invention provides a refrigerator. The refrigerator includes a housing, a screen, a plurality of buttons, a thermometer, and a central controller, wherein the housing has an inner chamber, the screen is disposed on an outer surface of the housing, the buttons are disposed on the housing, the thermometer is disposed inside the inner chamber of the housing to detect a temperature inside the inner chamber of the housing, and the central controller electrically connects the screen, the buttons, and the thermometer to control the temperature inside the inner chamber according to a predetermined temperature inputted via the buttons and to drive the screen to display the temperature inside the inner chamber detected by the thermometer. The refrigerator further comprises two body fat detecting buttons, an impedance detecting circuit, an analog-to-digital converter, and a body fat meter, wherein the two body fat detecting buttons are disposed on the outer surface of the housing, the impedance detecting circuit connects to the two body fat detecting buttons to detect an impedance of a human body when the human body touches both of the two body fat detecting buttons, the analog-to-digital converter electrically connects to the impedance detecting circuit and receiving the impedance of the human body and converting the impedance of the human body to a digital data of the impedance of the human body, and the body fat meter electrically connects to the analog-to-digital converter and the central controller respectively, the body fat meter calculates a body fat rate according to the digital data of the impedance of the human body and transmits the body fat rate to the central controller for displaying on the screen. According to the present invention, the human body may be aware of his/her health condition by the body fat meter within the refrigerator, and therefore choose proper food for him/her.

The housing can be included a main housing body and a door covering the main housing body, and the screen and the two body fat detecting buttons can be respectively disposed on an outer surface of the door. The screen can be comprised a liquid crystal display (LCD), or a light emitting diode (LED) display, or an OLED.

Furthermore, on the present invention, the refrigerator can be further included a memory device to keep a historical record of the body fat rate. Accordingly, an embodiment of the present invention is directed to a refrigerator.

It is to be understood that both the foregoing general description and the following detailed description are exemplary and explanatory only and are not restrictive of the present invention, as claimed.

The accompanying drawings, which are incorporated in and constitute a part of this specification, illustrate several embodiments of the present invention and together with the description, serve to explain the principles of the present invention.

### BRIEF DESCRIPTION OF THE DRAWINGS

FIG. 1 is a perspective view of a refrigerator of the present invention;

FIG. 2 is a function block diagram of the refrigerator of the present invention.

### DETAILED DESCRIPTION OF THE PREFERRED EMBODIMENT

Reference will now be made in detail to present embodiments of the present invention, examples of which are illustrated in the accompanying drawings. Wherever possible, the same reference numbers will be used throughout the drawings to refer to the same or like parts.

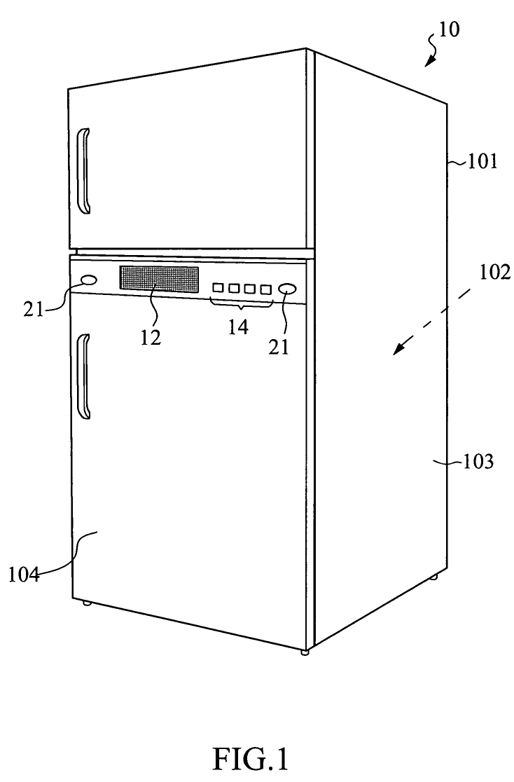

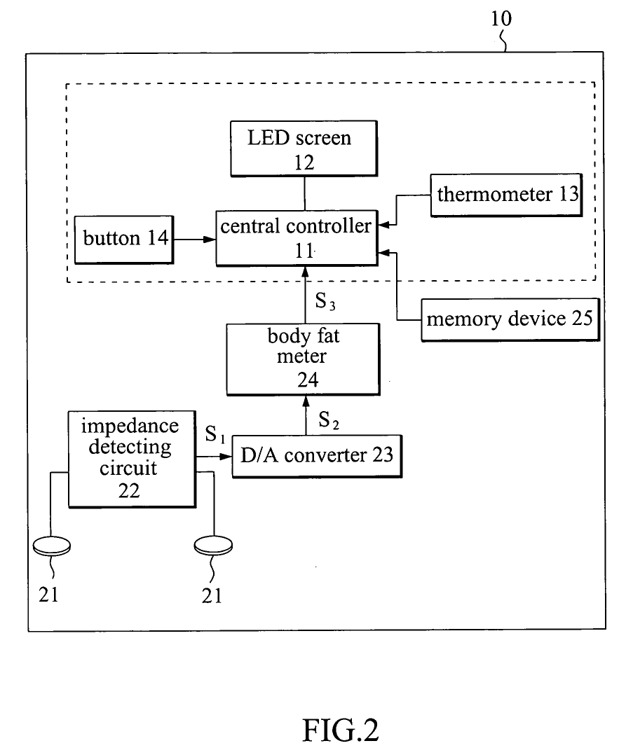

FIGS. 1 and 2 respectively show a perspective view and a function block diagram of a refrigerator of the present invention. The refrigerator includes a housing 101, a liquid crystal display (LCD) screen 12, a plurality of buttons 14, a thermometer 13, and a central controller 11, wherein the central controller 11 electrically connects the screen 12, the buttons 14, and the thermometer 13. The central controller 11 transmits and controls the screen 12 to display the temperature inside the housing 101 detected by the thermometer 13. The housing 101 has an inner chamber 102, and a human body S1 is capable of controlling a predetermined temperature inside the inner chamber 102 by the buttons 14. The refrigerator in the present invention further includes two body fat detecting buttons 21, an impedance detecting circuit 22, an analog-to-digital converter 23, and a body fat meter 24.

In the present embodiment, the housing 101 of the refrigerator includes a main housing body 103, a front door 104 covering the main housing body 103, and the body fat detecting buttons 21 are respectively disposed on the outer surface of the front door 104 or a side wall of the refrigerator. Furthermore, the buttons 14 may be also disposed on the outer surface of the front door 104. The impedance detecting circuit 22, which connects to the body fat detecting buttons 21, detects an electric current variation resulted from both of the body fat detecting buttons 21 pressed by the user and generates an impedance of the human body S1. A reaction time in the previous detecting step is not restricted. In the present embodiment, the reaction time is three seconds for preventing from calculating the body fat rate because of the human body S1 pressing the body fat detecting buttons 21 incidentally.

Moreover, the impedance detecting circuit 22 transmits the impedance of the human body S1 to the analog-to-digital converter 23. The analog-to-digital converter 23 electrically connects to the impedance detecting circuit 22 and converts the impedance of the human body S1 into a digital data of the impedance of the human body S2 and transmits the digital data of the impedance of the human body S2 to the body fat meter 24. The body fat meter 24 generates a body fat rate S3 corresponding to the digital impedance of the human body S2 and transmits the body fat rate S3 to the central controller 11 to display the body fat rate S3 on the screen 12. As showed in FIG. 2, the body fat rate S3 can be displayed on the screen 12, such as a liquid crystal display.

In the present embodiment, the refrigerator 10 further includes a memory device 25 to record the historical detected body fat rate S3. When the central controller 11 receives the body fat rate S3 and displays the body fat rate S3 on the screen 12, the memory device 25 keeps a historical record of the body fat rate S3. In addition, the user is capable of establishing a personal database by inputting the user's height and weight with the buttons 14. When the central controller 11 receives the body fat rate S3, the central controller 11 is capable of diagnosing whether the body fat rate S3 is normal and warning the user whose health condition.

As described above, the impedance detecting circuit 22, which connects the body fat detecting buttons 21, detects an electric current variation resulted from both of the body fat detecting buttons 21 pressed by the user and calculates an impedance of the human body S1. The body fat meter 24 generates a body fat rate corresponding to the impedance of the human body S1. And then the body fat rate is displayed on the screen. Therefore, the user may be aware of his/her health condition and choose some food that is suitable for his/her health condition.

Other embodiments of the present invention will be apparent to those skilled in the art from consideration of the specification and practice of the present invention disclosed herein. It is intended that the specification and examples be considered as exemplary only, with a true scope and spirit of the present invention being indicated by the following claims.

What is claimed is:

1. A refrigerator, comprising:
a housing having an inner chamber;
a screen disposed on an outer surface of said housing;
a plurality of buttons disposed on said housing;
a thermometer disposed inside the inner chamber of said housing and detecting a temperature inside the inner chamber of said housing;
a central controller electrically connecting to said screen, said buttons, and said thermometer to control the temperature inside said inner chamber according to a predetermined temperature inputted via said buttons and to drive said screen to display the temperature inside the inner chamber detected by said thermometer;
two body fat detecting buttons disposed on the outer surface of said housing;
an impedance detecting circuit connecting to said two body fat detecting buttons to detect an impedance of a human body when said human body touches both of said two body fat detecting buttons;
an analog-to-digital converter electrically connecting to said impedance detecting circuit and receiving the impedance of said human body and converting the impedance of said human body to a digital data of the impedance of said human body; and
a body fat meter electrically connecting to said analog-to-digital converter and said central controller respectively, said body fat meter calculating a body fat rate according to the digital data of the impedance of said human body, and transmitting said body fat rate to said central controller for displaying on said screen.

2. The refrigerator of claim 1, wherein said housing comprises a main housing body and a door covering said main housing body.

3. The refrigerator of claim 2, wherein said screen and said two body fat detecting buttons are disposed on an outer surface of said door respectively.

4. The refrigerator of claim 1 further comprising a memory device keeping a historical record of said body fat rate.

5. A refrigerator of claim 1, wherein said screen comprises a liquid crystal display (LCD).
